(19) United States
(12) Patent Application Publication
(10) Pub. No.: US 2024/0026276 A1
(43) Pub. Date: Jan. 25, 2024

(54) SENSING APPARATUS AND METHOD

(71) Applicant: UNIVERSIDADE DE COIMBRA, Coimbra (PT)

(72) Inventors: Paulo Roberto FERREIRA DA ROCHA, Coimbra (PT); David TOSH, Bath (GB)

(21) Appl. No.: 18/255,458

(22) PCT Filed: Dec. 2, 2021
(86) PCT No.: PCT/IB2021/061252

§ 371 Date: Jun. 1, 2023

(30) Foreign Application Priority Data
Dec. 2, 2020 (GB) 2018980.9
Nov. 24, 2021 (PT) 117602

(51) Int. Cl. C12M 1/34; C12M 1/12; C12M 1/00; C12M 3/00; C12M 1/42

(52) U.S. Cl. CPC C12M 41/46; C12M 25/02; C12M 23/34; C12M 23/44; C12M 35/02

(57) ABSTRACT

A method of providing sensing apparatus and a sensing method are disclosed. One aspect provides sensing apparatus comprising: a chamber configured to receive a biological sample at least part of a wall of the chamber comprising a printed circuit board substrate; the part of the wall comprising a printed circuit board substrate comprising an electrode configured to transmit or receive a signal to the biological sample, the electrode being coupleable to a sensing control unit located outside the chamber. The apparatus comprising a channel in communication with the chamber, the channel being dimensioned to allow at least a portion of the biological sample extend through the channel, the channel comprising a conductive surface configured to transmit or receive a signal to at least a portion of the biological cell sample extendable through the channel, the conductive channel surface being coupleable to the sensing control unit located outside the chamber.

### TECHNICAL FIELD

The present disclosure relates to aspects and embodiments related to sensing apparatus, a method of providing sensing apparatus and a sensing method.

One aspect provides sensing apparatus comprising: a chamber configured to receive a biological sample at least part of a wall of the chamber comprising a printed circuit board substrate; the part of the wall comprising a printed circuit board substrate comprising an electrode configured to transmit or receive a signal to the biological sample, the electrode being coupleable to a sensing control unit located outside the chamber. The apparatus comprising a channel in communication with the chamber, the channel being dimensioned to allow at least a portion of the biological sample extend through the channel, the channel comprising a conductive surface configured to transmit or receive a signal to at least a portion of the biological cell sample extendable through the channel, the conductive channel surface being coupleable to the sensing control unit located outside the chamber.

### BACKGROUND

Understanding the operation of a brain represents a major scientific challenge. Behaviour, memory, emotion, learning ability, cognitive dysfunction and other forms of dementia appear to rely upon communication pathways within a brain. Cell to cell signalling is believed to play a fundamental role in brain operation and disease pathogenesis.

Bioelectronics: a convergence between biology and electronics, represents one route which is allowing for exploration of structure and function of a brain. Bioelectronics can, for example, enable study of brain cells via an understanding of electrical signals supported by those cells. In particular, electrical signals can, for example, be applied to cells and recorded to inform understanding of brain cell operation, response to stimuli, disease diagnosis and/or applied to cells in a manner which is modulated to effect disease treatment.

Bioelectronics may offer a mechanism by which it is possible to accurately translate communication between brain cells, nerves and brain tissue into a readable, reproducible and comprehensive language.

These facts are disclosed in order to illustrate the technical problem addressed by the present disclosure.

### General Description

A first aspect provides sensing apparatus comprising: a chamber configured to receive a biological sample; at least part of a wall of the chamber comprising a printed circuit board substrate; the part of the wall comprising a printed circuit board substrate comprising an electrode configured to transmit or receive a signal to the biological sample, the electrode being coupleable to a sensing control unit located outside the chamber; a channel in communication with the chamber, the channel being dimensioned to allow the biological sample to extend through the channel, the channel comprising a conductive surface configured to transmit or receive a signal to at least a portion of the biological sample extendable through the channel, the conductive channel surface being coupleable to the sensing control unit located outside the chamber.

Aspects recognise that the electrical nature of some biological material, including, for example, excitable cells such as neurons, can allow for study of the operation of such biological material. By way of example, brain circuit models have been developed to describe the physiological processes underlying human behaviours and understanding correlation between a “biological circuit” and brain behaviour can allow for a more detailed investigation of cellular processes occurring in communicating nerve cells in specific brain areas. One example of brain circuit-based research is the Research Domain Criteria (RDoC) approach according to which, neurological and psychiatric symptoms are considered to be a result of one or more malfunctioning brain circuit.

More generally, aspects recognise that study of a biological sample can be of interest when determining or studying the functionality and/or behaviour of that biological sample. Aspects provide a mechanism to study a biological sample when housed in a chamber and as that sample interacts in a controlled manner via a link with another environment. The other environment may, for example, comprise a further biological sample, or may, for example, comprise a chemical or other environment which may cause a response in the biological sample under study.

In the case of excitable cells which themselves have an electrical response related to function and behaviour, it will be appreciated that the operation of excitable cells within an organism depends upon ion fluctuations leading to events such as action potentials. Action potentials are spikes in voltage or membrane potential across a cellular membrane. Concentration of ions in an excitable cell allow for generation of current. Sufficient current is required to initiate a voltage response in a cell membrane; if the current is insufficient to depolarize the membrane to a threshold level, an action potential will not fire. Various excitable cells operate as a result of action potentials including, for example, neurons and muscle cells. Other biological cells, tissues and organisms, including, for example, glia cells and their transformed counterparts glioma, cancerous cells, including breast cancer and prostate cancer cells and similar have also been reported to manifest ion gradients leading to electrical pulses.

Aspects recognise that providing a mechanism to analyse all or part of the function of a excitable cell circuit, for example, a brain cell circuit, can give access to information regarding neuronal subtypes involved. Furthermore, by allowing analysis of neurons at a molecular level, identification of novel targets for treatment of neurological or psychiatric symptoms may be possible.

One method to explore excitable cell to cell signalling is electrophysiology. Electrophysiological approaches are typically supported by use of microelectrode arrays (MEAS) to record extracellular activity of electrogenic cells. Microelectrode arrays comprise 2D planar electrodes arranges on a substrate in close contact with relevant cells under study maintained in culture medium. MEAs are configured to detect extracellular field potential, which, in the case of neurons, is a superposition of action potentials of individual neurons, through synaptic potentials, to glial potentials. Typically, MEA technology is unable to support measurement of electrical activity of well-defined cell populations. The electrical recordings of separate brain regions and communication between cell populations is not supported by known MEAs.

The first aspect recognises that by providing a primary chamber in which cells are housed and a monitorable channel through which cells may extend, it may be possible to study interaction of a population of cells with another population of cells.

Accordingly, sensing apparatus according to the first aspect may comprise a substrate. That substrate may be any appropriate substrate upon which appropriate components may be assembled or formed. The apparatus may comprise a chamber located on, or integrally formed as part of, the substrate. The chamber may be configured to receive a biological sample. The biological sample may comprise a simple biological organism, a biological tissue sample, or cultured sample, a cell population or biological cell sample. The biological sample may comprise an organism, tissue, cell population or cells which exhibit an electrical response which correlates with one or more aspect of their biological function. The biological sample may comprise one or more cells which exhibit a response, for example an electrical or thermal response, detectable by electrodes, which correlates with one or more aspect of their biological function. The biological sample may, for example, comprise excitable cells. The excitable cells may, for example, comprise brain cells such as neurons, glial cells and similar. The biological sample may comprise a fluid including one or more cells. The biological sample may be adherable to at least one inner surface of the chamber. The biological sample may comprise a fluid including a single cell type, a tissue formed from a single cell type, a cell population of a single cell type, and/or a single cell type.

At least part of a wall of the chamber may comprise a printed circuit board substrate. The printed circuit board substrate may include an electrode, or a plurality of electrodes configured to transmit or receive a signal to the biological sample. The conductive electrode may therefore be located on or formed on a wall of the chamber. The conductive electrode may be electrically and/or thermally conductive. The conductive electrode may be configured to sense or stimulate a biological sample locatable within the chamber. The apparatus may include a plurality of conductive electrodes located on or formed on a wall of the chamber. The plurality of conductive electrodes may comprise a multi-electrode array. In some embodiments, the apparatus may include an electrically conductive sample sensor located on a wall of the chamber. The apparatus may include a plurality of electrically conductive sample sensors. The electrically conductive sample sensors may comprise a multi electrode array of sample sensors. The electrode(s) and/or sensor(s) may be configured to transmit an electrical signal into the chamber. One or more electrode(s) and/or sensor(s) may be configured to receive an electrical signal(s) generated by a biological sample located within the chamber, or an indication of an electrical property associated with the biological sample located within the chamber. The electrode(s) and/or sensor(s) may be connected to an electrical connector located outside the chamber. The electrical connector may be coupleable or directly connectable to an electrical source or appropriate electrical sensing device, and/or to a sensing control unit located outside the chamber.

The sensing control unit may be configured to monitor and/or control operation of the components of the printed circuit board and/or further active components of a device of which, in use, the sensing apparatus forms part.

The apparatus may comprise a channel in communication with the chamber. The channel may be dimensioned to allow at least a portion of the biological sample to extend through the channel. The channel may be dimensioned to allow a portion of the biological sample to extend through the channel. The channel may be dimensioned to allow several cells, only a single cell or part of a cell to extend through the channel. The channel may be shaped and/or dimensioned to prevent flow of the biological sample from the chamber through the channel. The channel may comprise a conductive surface. That conductive surface may comprise a conductive layer or coating. The conductive inner surface may be configured to transmit or receive an electrical or thermal signal to at least a portion of a biological sample which extends through the channel. The conductive channel inner surface may be connected or to a further electrical connector located outside the chamber and outside the channel. The further electrical connector may be coupleable, to an electrical source and/or sensing device and/or sensing control unit located outside the chamber, thus allowing electrical or thermal signals within the channel to be detected, or electrical or thermal signals to be passed to the conductive surface of the channel.

The electrical or thermal signal received or transmitted by the electrode or conductive surface may comprise an indication of a property of a biological sample locatable within the chamber. The property may, for example, comprise: sample impedance, current, voltage, temperature, and may correlate to another function of the biological sample under study.

In some embodiments, at least one complete wall of the chamber comprises a printed circuit board substrate, and one or more of: the electrode, channel, and conductive surface are integrally formed on the PCB substrate.

Accordingly, use of a PCB substrate can allow for simple, efficient and cost-effective manufacture of sensing apparatus in accordance with the first aspect. Rather than assembling each component separately, an integral manufacturing process may be utilised. Printed Circuit Boards (PCB) represent an established technology providing an inexpensive, robust and well-understood basis upon which arrangements can be built. PCB miniaturization is possible by use of multilayer rigid or flexible boards. Such miniaturisation, combined with an ability to construct PCBs using biocompatible materials, makes PCB fabrication technology a strong candidate to support excitable cell modelling and study platforms.

In some embodiments, one or more of: the substrate, chamber, sample sensor, channel, and conductive inner surface are, at least in part, constructed from, or include a biocompatible material. In some embodiments, at least a surface portion of the substrate, chamber, sample sensor, channel, and conductive inner surface which, in use, may contact the biological sample is formed from a biocompatible material. Accordingly, biological samples located on or in contact with such components may maintain viability for a duration of time sufficient to support study of such cells. By way of example, biocompatible base materials for flexible PCB construction, for example, span polyester (PET), polyimide (PI), polyethylene naphthalate (PEN), polyetherimide (PEI), and various fluropolymers (FEP) and copolymers. Thin and noble-metal electroplated copper films have flexible properties and can also offer appropriate biocompatibility, whilst maintaining conductive properties where appropriate.

In some embodiments, the channel may comprise a via in a PCB layer or substrate. A via is an electrical connection between layers of a PCB. A via or channel in accordance with aspects and embodiments may comprise a physical channel or opening linking one or more layers or one or more chambers provided. A via or channel in accordance with embodiments may be located or constructed through a plane of one or more adjacent substrate layers. Appropriate use of processes, such as through hole connections and wet process chemistry, are such that vias and electrodes can be constructed, functionalized and rationalized down to appropriate dimensions to support biological sample study. For example, some techniques may support construction of a via having a cross sectional area of around 50 μm2, thereby supporting the growth of biological cells through such channels or vias.

In some embodiments, at least one wall of the chamber comprises an adhesion coating layer, such as poly(lysine) (PL), poly(ornithine) (PO), poly(arginine), poly(ethylenimine) (PEI), poly-L-lysine (PLL), poly-D-lysine (PDL), poly-L-ornithine (PLO), extracellular cell matrix or a laminin coating. In some embodiments, the conductive channel inner surface comprises a laminin coating. Provision of a laminin coating may assist with biocompatibility and ensure that the viability of cells in a biological sample may be extended compared to an arrangement in which no adhesion coating layer is provided. Such a coating may assist cells in a biological sample with adhesion to a surface of a chamber. Such adhesion may assist if the sensors are provided in the same region as adhesion is desired, since greater sensitivity to electrical detection and/or stimulation of the biological sample may be achievable.

In some embodiments, at least one wall of the chamber is enriched with a cell growth factor, for example, FGF2 (Fibroblast growth factor 2) or Nerve Growth Factor. Accordingly, growth of cells in a biological sample may be encouraged between chambers, across a chamber, into a channel and similar, as desired or envisaged for a particular sensing apparatus application.

In some embodiments, the apparatus comprises: a second chamber configured to receive a second biological sample; at least part of a wall of the second chamber comprising a printed circuit board substrate; the part of the wall of the second chamber comprising a printed circuit board substrate comprising a second chamber electrode configured to transmit or receive a second chamber signal to the second biological sample the second chamber electrode being coupleable to a sensing control unit located outside the second chamber, and wherein the channel connects the chamber and the second chamber. Accordingly, it is possible to provide a sensing apparatus comprising one or more compartments or chambers each configured to house a biological sample. One or more of the compartments may be directly connectable and/or in communication with one or more other compartment. The connection may occur through appropriate location of one or more channels or vias. The connection or communication may occur via a physical link between biological samples housed in separate discrete chambers or compartments.

In some embodiments, the printed circuit board substrate comprising the at least part of a wall of the chamber and the printed circuit board substrate comprising the at least part of a wall of the second chamber are the same printed circuit board substrate.

In some embodiments, the printed circuit board substrate comprising the at least part of a wall of the chamber and the printed circuit board substrate comprising the at least part of a wall of the second chamber are distinct printed circuit board substrates.

In some embodiments, the apparatus comprises: at least one further substrate located above or below the substrate, and wherein the chamber is formed between the substrate and the further substrate. In some embodiments, the second chamber is also formed between the substrate and the further substrate. In some embodiments, the second chamber is formed between the substrate and a different further substrate to the further substrate and substrate forming the chamber. The compartments may be formed, for example, on or between one or more PCB layers. The layers may form a multi-layer PCB. Coupling or connection between compartments may be as a result of appropriately formed vias between PCB layers.

In some embodiments, the apparatus comprises an access port configured to allow insertion of the biological cell sample into the chamber. In some embodiments, the apparatus comprises: an access port associated with each chamber, each access port being configured to allow insertion of a biological cell sample into the respective chamber. In some embodiments, the chamber and channel are in communication with each other and a cell culture media source. Accordingly, conditions to maintain viability of biological cell samples may be maintained or adjusted with ease. Insertion of different chemical stimulus may be enabled via access ports.

In some embodiments, the sensing apparatus may form part of a larger sensing device comprising, for example a sensing control unit, culture environment control elements including, for example culture media configured to pass through the chamber, together with associated flow control elements, temperature control sensors and control mechanisms and similar.

In some embodiments, the channel of the sensing apparatus is coupleable with, or connectable to, another sensing apparatus. Accordingly, the sensing apparatus may be substantially modular, allowing for creation of a more complex sensing apparatus arrangement by appropriate coupling of two or more sensing apparatus according to the first aspect.

A second aspect provides a method of forming sensing apparatus, the method comprising: providing a chamber in which at least part of a wall of the chamber comprises a printed circuit board substrate, wherein the part of the wall comprising a printed circuit board substrate comprises an electrode configured to transmit or receive a signal to a biological sample locatable within the chamber, the electrode being coupleable to a sensing control unit located outside the chamber; and providing a channel in communication with the chamber, the channel being dimensioned to allow the biological sample to extend through the channel, the channel comprising a conductive surface configured to transmit or receive a signal to at least a portion of the biological sample extendable through the channel, the conductive channel surface being coupleable to the sensing control unit located outside the chamber.

In some embodiments, the method comprises arranging the printed circuit borad substrate so that at least one complete wall of the chamber comprises a printed circuit board substrate, and integrally forming one or more of: the electrode, channel, and conductive surface on the PCB substrate.

In some embodiments, the method comprises constructing one or more of: the substrate, chamber, electrode, channel, and conductive surface such that they include a biocompatible material.

In some embodiments, the method comprises providing at least one wall of the chamber with an adhesion coating layer. In some embodiments, the adhesion coating layer comprises one of: poly(lysine) (PL), poly(ornithine) (PO), poly(arginine), poly(ethylenimine) (PEI), poly-L-lysine (PLL), poly-D-lysine (PDL), poly-L-ornithine (PLO), extracellular cell matrix or laminin.

In some embodiments, the method comprises providing the conductive channel surface with an adhesion coating layer. In some embodiments, the adhesion coating layer comprises one of: poly(lysine) (PL), poly(ornithine) (PO), poly(arginine), poly(ethylenimine) (PEI), poly-L-lysine (PLL), poly-D-lysine (PDL), poly-L-ornithine (PLO), extracellular cell matrix or laminin.

In some embodiments, the method comprises enriching at least one wall of the chamber with a cell growth factor. In some embodiments, the cell growth factor comprises one of: FGF2 (Fibroblast growth factor 2) or Nerve Growth Factor.

In some embodiments, the method comprises: providing a second chamber configured to receive a second biological sample; at least part of a wall of the second chamber comprising a printed circuit board substrate; the part of the wall of the second chamber comprising a printed circuit board substrate comprising a second chamber electrode configured to transmit or receive a second chamber signal to the second biological sample the second chamber electrode being coupleable to a sensing control unit located outside the second chamber, and wherein the channel connects the chamber and the second chamber.

In some embodiments, the method comprises arranging the substrates such that the printed circuit board substrate comprising the at least part of a wall of the chamber and the printed circuit board substrate comprising the at least part of a wall of the second chamber are the same printed circuit board substrate.

In some embodiments, the method comprises arranging the substrates such that the printed circuit board substrate comprising the at least part of a wall of the chamber and the printed circuit board substrate comprising the at least part of a wall of the second chamber are distinct printed circuit board substrates.

In some embodiments, the method comprises: locating at least one further substrate above or below the substrate, such that the chamber is formed between the substrate and the further substrate.

In some embodiments, the second chamber is also formed between the substrate and the further substrate.

In some embodiments, the second chamber is formed between the substrate and a different further substrate to the further substrate and substrate forming the chamber.

In some embodiments, the method comprises providing an access port configured to allow insertion of the biological sample into the chamber.

In some embodiments, the method comprises proving an access port associated with each chamber, each access port being configured to allow insertion of a biological sample into the respective chamber.

In some embodiments, the chamber and channel are in communication with each other and a cell culture media source.

In some embodiments, the channel of the sensing apparatus is coupleable with another sensing apparatus.

A third aspect provides a sensing method comprising: inserting a biological sample into the chamber of sensing apparatus according to the first aspect; monitoring properties of the biological sample in the chamber and channel using the electrode and conductive surface of the channel and the sensing control unit.

In some embodiments, the method comprises: electrically stimulating the biological sample in the chamber using the electrode and an electrical source; and monitoring a response of the biological sample using a further electrode and the sensing control unit.

In some embodiments, the method comprises: chemically stimulating the biological sample in the chamber and monitoring a response of the biological sample using the electrode and the sensing control unit.

A further aspect provides sensing apparatus comprising: a substrate; a chamber on the substrate configured to receive a biological cell sample; an electrically conductive sample sensor located on a wall of the chamber configured to transmit or receive an electrical signal to the biological cell sample and coupleable to an electrical connector located outside the chamber, the electrical connector being connectable to an electrical source or sensing device; a channel in communication with the chamber, the channel being dimensioned to allow the biological cell sample to extend through the channel, the channel comprising a conductive inner surface configured to transmit or receive an electrical signal to a portion of the biological cell sample extending through the channel, the conductive channel inner surface being coupleable to a further electrical connector located outside the chamber, the further electrical connector being connectable to an electrical source or sensing device.

A further aspect provides a method of forming sensing apparatus, the method comprising: providing a substrate; providing a chamber on the substrate and configuring the chamber to receive a biological cell sample; locating an electrically conductive sample sensor on a wall of the chamber and configuring the sample sensor to transmit or receive an electrical signal to the biological cell sample, locating an electrical connector, coupleable to the sensor, outside the chamber, the electrical connector also being connectable to an electrical source or sensing device; providing a channel in communication with the chamber, the channel being dimensioned to allow the biological cell sample to extend through the channel, the channel comprising a conductive inner surface configured to transmit or receive an electrical signal to a portion of the biological cell sample extending through the channel, the conductive channel inner surface being coupleable to a further electrical connector located outside the chamber, the further electrical connector being connectable to an electrical source or sensing device.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

Where elements are described as being connected or connectable, they may be directly connected. Where elements are described as being coupled or coupleable, they may be linked by one or more intervening or interposing elements.

### BRIEF DESCRIPTION OF THE DRAWINGS

The following figures provide preferred embodiments for illustrating the disclosure and should not be seen as limiting the scope of invention.

FIG. 1 illustrates schematically a cross section of some main components of a sensor apparatus of one arrangement;

FIG. 2 illustrates schematically an exploded perspective view of a possible apparatus arrangement;

FIGS. 3a to 3f provides an illustration of cell viability when subjected to differing base materials;

FIG. 4a is a representation of a possible 3 layer PCB stack which forms apparatus according to described arrangements; and

FIG. 4b is a representation of some components used to form a PCB stack such as that shown in FIG. 4a. Schematic representation of an embodiment of a the present disclosure.

FIG. 5a-5c Schematic representation of an embodiment of PCB stack of the present disclosure.

### DETAILED DESCRIPTION

### Biological Background

Arrangements facilitate in vitro, electrophysiological-based, study of excitable cells. Understanding the operation of excitable cells can facilitate further research. For example, in the case of brain cells, including, for example, neurons and glial cells, study of communication between such cells, or cell populations, may provide visibility of neuronal subtypes involved in communications and consequently an indication of possible focus areas for treatment of diseases or malfunctions of the brain. In the case of brain cells, arrangements provide a mechanism to locally and non-invasively study the interaction of the types of cells which can form specific brain regions. Arrangements allow the identification of novel targets for treatment of, for example, cognitive dysfunctions, neurological and psychiatric disorders by providing a method to access communication processes of nerve cells in and between specific brain areas, in a precise, real-time and non-invasive way.

Analogous approaches to those described below can be applied in relation to other excitable cells, including cells forming cell populations or tissue as appropriate.

### Technological Background

Printed Circuit Boards (PCB) represent an established technology providing an inexpensive, robust and well-understood basis upon which arrangements can be built. PCB miniaturization is possible by use of multilayer rigid or flexible boards. Such miniaturisation, combined with an ability to construct PCBs using biocompatible materials, makes PCB fabrication technology a strong candidate to support excitable cell modelling and study platforms. Biocompatible base materials for flexible PCB construction, for example, span polyester (PET), polyimide (PI), polyethylene naphthalate (PEN), polyetherimide (PEI), and various fluropolymers (FEP) and copolymers. Thin and noble-metal electroplated copper films have flexible properties and can also offer appropriate biocompatibility.

A via is an electrical connection between layers in a PCB. A via may be located or constructed through a plane of one or more adjacent layers. Appropriate use of processes such as through hole connections and wet process chemistry, vias and electrodes can be constructed, functionalized and rationalized down to a size of 50 μm².

Before describing particular implementations in detail, a general overview is provided.

### Overview

Arrangements provide a novel sensor based on PCB technology that enables accurate modelling of cell to cell signalling in a laboratory. The cells under study may comprise any appropriate excitable cell, and cells having electrical characteristics which are indicative of cell operation and function. In particular, arrangements provide apparatus which facilitates in vitro study of brain cells.

Arrangements provide a sensing apparatus comprising one or more compartments each configured to house a cell population. One or more of the compartments may be connectable or in communication with one or more other compartment. That connection or communication may occur via a physical link between compartments. The compartments may be formed on one or more PCB layers. The layers may form a multi-layer PCB. Coupling or connection between compartments may be as a result of appropriately formed vias between PCB layers. The compartments may include one or more electrical connection or electrodes, configured to allow electrical stimulation or measurement of a cell population located within the compartment.

Arrangements recognise that differentiating cells from human tissue can provide a realistic model for human biology. For example, neural stem cells can be differentiated into specific brain cells in-vitro. It is expected that more cells will be found and defined protocols established suitable for differentiation in-vitro. A sensing apparatus according to arrangements is configured to support the differentiation and culture of appropriate cells to be studied within one or more provided compartments.

For example, in arrangements which generally comprise a sensing apparatus in the form of a multilayer PCB, there are provided multiple PCB layers arranged in a stack. Each layer is prepared such that it has a well-defined cell population in one or more compartment provided on that layer. The cell population is adhered to multiple conducting electrodes. The conducting electrodes are distributed on the surface of each layer so that the electrical monitoring and stimulus of the defined cell populations in the compartment can be achieved. For example, within a compartment or chamber, an MEA array comprising gold-plated cell activity sensing or stimulation electrodes can be formed. That array and chamber can be formed on a biocompatible PCB core.

Conducting vias are provided between compartments in a layer and between layers of a multi-layer arrangement. The conducting vias provide a physical and electrical connection between layers in a stacked PCB apparatus. The vias can serve as electrodes providing unique connection points to record and stimulate cell-cell communication between different brain regions with a single nerve precision. Vias facilitate provision of localized connection points between different cell populations. Those cell populations may be representative of different brain regions. Consequently, the vias create a unique opportunity to facilitate study of communication pathways between cell populations.

The MEA array and conducting vias enable real-time monitoring of specific cell activity, as cells are exposed to stimulus. The stimulus may comprise chemical or electrical stimulus. Chemical cell stimulus may be applied to cells under study by, for example, the passing of an appropriate chemical fluid through one or more of the compartments or chambers housing cells. Flow of fluid through the sensing apparatus can be facilitated by appropriate positioning of vias. Similarly, electrical stimulation may be implemented in arrangements by location of appropriate electrodes and connection points. Arrangements may be designed and formed such that electrode connections are routed in a conventional, slot-type edge connector. Such a configuration can allow for effortless electronic instrumentation interfacing.

Electrical and chemical stimulation of cells may occur over a period of time. Electrical monitoring of cells located within the sensor apparatus of arrangements can also span long time periods. In order to monitor live cells, those cells housed in and on the PCB chambers and compartments can be exposed to appropriate conditions to try and ensure cell longevity. In particular, the sensor apparatus and PCB components may be formed from biocompatible materials. Furthermore, if long-term study of cells is desired, cells may be provided with appropriate nutrient and environmental conditions to support cell survival. For example, a PCB module can be inserted inside an incubator at 37 degrees centigrade and having an ambient surrounding atmosphere of 5% CO₂. A miniaturized PCB-based incubator, with heaters, temperature and CO2 controls can also be derived.

It will be appreciated that various parameters of the sensor apparatus may be adjusted to suit an envisaged application. In particular, the number of PCB layers, the shape of the PCB, the location and number of compartments, the location and number of vias and similar can all be easily modified using conventional PCB manufacturing techniques. Those techniques include, for example, panel preparation, chemical etching, drilling, photo polymerization, plating, pressing and lamination.

For example, if undertaking a study of brain cells, the structure of the sensing apparatus may be selected based upon a region of brain under study. By way of example, the number of layers in the structure may be selected in dependence upon the complexity of the brain region under study. A sensor apparatus comprising a PCB module can be created to allow monitoring and stimulation of cell to cell communication between different brain regions of the human brain. The monitoring of different brain region interaction is done via specific interlayer bridges, in the form of appropriately located vias. It will be appreciated that some arrangements allow for a single PCB module to be expanded by providing additional PCB modules, which may be identical or may differ from the first PCB module. Interconnection of PCB modules containing brain cells can allow for creation of part, or all, of a human brain to be modelled in-vitro.

In general, the described PCB compartment/chamber architecture can be selected or designed in dependence upon the excitable cell(s) of interest. As described above, for example, the structure can be selected based upon a specific type of brain cell(s) under study. One or more PCB layers having chambers/compartments can be stacked to form a multilayer, 3D structure. The cell types and connections between chambers/compartments can be arranged in order to model one or more parts of a brain.

Having provided a general overview of possible arrangements, a selection of possible implementations are now described in more detail.

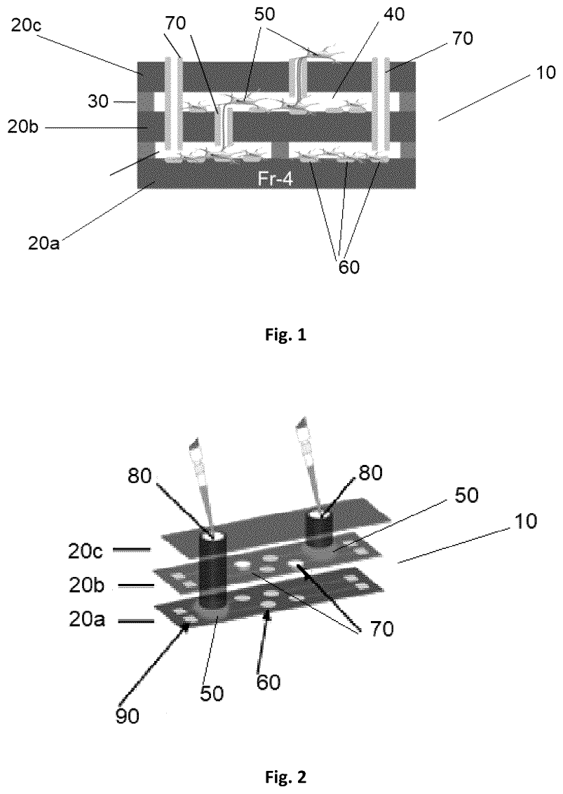

FIG. 1 illustrates schematically a cross section of some main components of a sensor apparatus of one arrangement. The apparatus 10 shown in FIG. 1 comprises: three PCB layers 20a, 20b, 20c affixed together by appropriate adhesive 30. The stacking of layers 20a, 20b, 20c forms chambers/compartments 40 configured to house brain cells 50. Each chamber includes a multi electrode array 60 formed on the surface of a chamber wall. Chambers and layers are connected physically and/or electrically by one or more vias 70.

In the example shown schematically in FIG. 1, cell culture chambers 40 for different brain cell types (for example, neurons, glia and similar) are provided. The chambers are electrically and physically connected through the vias 70. Such a physical connection allows physical expansion and interaction between different cell types housed in chambers 40. The electrical connection allows for electrical examination of any signalling associated with physical interaction. Provision of multi electrode arrays 60 in each chamber allows for specific electrical monitoring within the individual cell chambers before, during, and after any physical interaction between cells housed in different chambers. The structure is arranged such that an identical extracellular environment can be provided for all chambers housing cells.

Although not shown in FIG. 1, it will be understood that one or more vias 80 having a larger diameter can be provided in relation to each layer of apparatus 10. The large via can be dimensioned to allow for cell medium change and cell deposition.

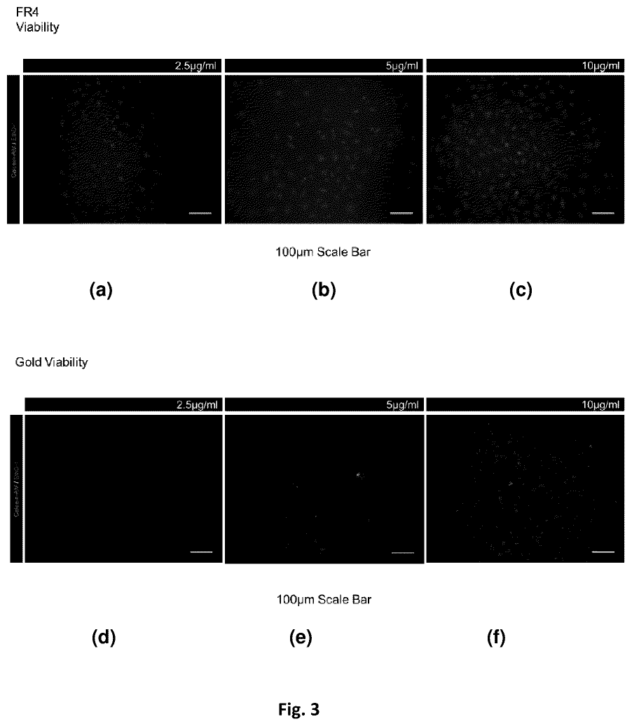

FIG. 2 illustrates schematically an exploded perspective view of a possible apparatus arrangement. As in FIG. 1, apparatus 10 is provided. The apparatus comprises three PCB layers: bottom layer 20a, middle layer 20b and access layer 20c. Large access vias 80 are shown. One provides access to the middle layer and one provides access to the bottom layer 20a. Cells to be grown in chambers on the bottom and middle layers can be introduced and maintained through vias 80. Smaller vias 70 are provided to connect cell chambers housing cells 50 on the middle and bottom layers. Each cell chamber includes a multi electrode array for sensing electrical activity within the cell population housed in a given chamber. Connector pads 90 are provided to allow recording of electrical signals detected by the MEA 60. Apparatus such as that shown in FIG. 2 may comprise 3 FR4 thin layers with gold (Au) plated electrodes, vias and traces. In such an example, typical dimensions of a layer may be 30 mm×20 mm, having a trace width of 0.127 mm, via diameter 0.2 mm and FR4 layer thickness of 0.2 mm.

### Construction

Material choices for construction of apparatus such as that shown in FIGS. 1 and 2 are determined by biocompatibility and established PCB construction methods. PCB base materials such as, for example, glass-reinforced epoxy laminate and FR-4 are suited to construction of apparatus such as those shown in FIGS. 1 and 2. Electrode material, for example, such as Au, and copper, once plated with Au, are suitably biocompatible. Of particular relevance is the likely longevity of cells placed in the chambers of the apparatus and therefore material choice can be based upon suitable levels of determined biocompatibility. In order to aid imaging of a biological sample, for example, microscopy of various types, the soldermask colour of the PCB can be chosen appropriately. In one example, the soldermask colour of the PCB may be black.

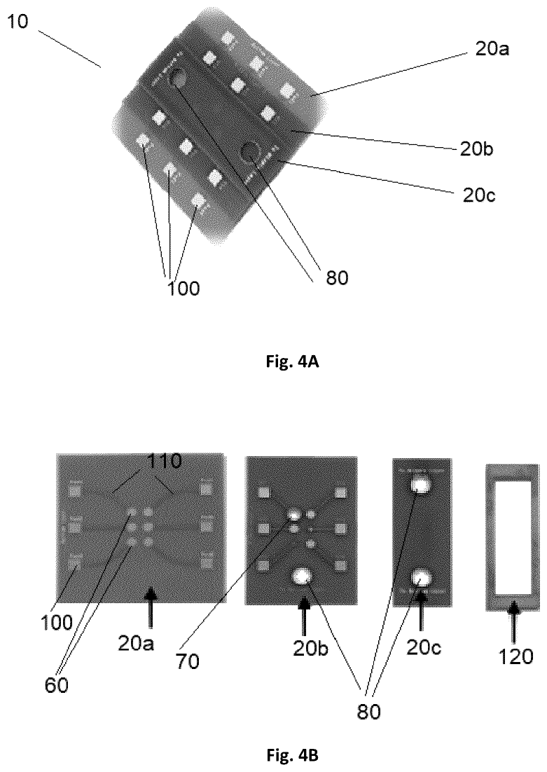

FIGS. 3a to 3f provides an illustration of cell viability when subjected to differing base materials. An indication of cell viability for human relevant induced pluripotent stem cell (iPSC) derived midbrain dopaminergic neurons (mDANs) under Fr4 and Cu/Au having different laminin cell concentrations was studied. FIGS. 3a to 3c provides an indication of cell survival via fluorescence. FIG. 3a shows cell viability on FR4 having a 2.5 microgram per millilitre laminin coating; FIG. 3b shows cell viability on FR4 having a 5.0 microgram per millilitre laminin coating; FIG. 3c shows cell viability on FR4 having a 10.0 microgram per millilitre laminin coating. It can be seen that an arrangement of cells on FR4 having a 10 ug/ml laminin coating show good survival rate (indicated by the degree of green florescence).

Surprisingly, FIGS. 3d to 3f appear to indicate that cell viability on a portion of PCB having an Au coating, for example, electrodes forming part of the multielectrode array provided in each chamber, is compromised. FIG. 3d shows cell viability on a gold coating having a 2.5 microgram per millilitre laminin coating; FIG. 3e shows cell viability on gold having a 5.0 microgram per millilitre laminin coating; and FIG. 3f shows cell viability on gold having a 10.0 microgram per millilitre laminin coating. These results are informative and indicate that unless the copper forming part of a PCB can be appropriately and consistently coated with gold, a “gold” portion of a PCB shows weak cell survival due to copper exposure. If gold is to be used, it is important to optimise electrochemical Au deposition to properly coat the Cu and then ensure that the cells to be studied adhere reliably to a more uniform surface of Au coated with laminin. Such an approach can help ensure cell viability within each chamber.

Alternative base materials to FR-4 include, for example: polyester (PET), polyimide (PI), polyethylene naphthalate (PEN), polyetherimide (PEI), along with various fluropolymers (FEP) and copolymers. Alternative electrode materials to Au include, for example: poly-3,4-ethylenedioxythiophene (PEDOT)—carbon nanotube (CNT), poly(3,4-ethylenedioxythiophene) polystyrene sulfonate, Platinum (Pt) black and titanium nitride (TiN). Extracellular electrodes, field effect transistors, rounded shape and square shaped electrodes may also have application within arrangements and their biocompatibility may need to be considered.

Vias are physical connection between layers. They provide the unique connection points between biological samples adhered to chambers in each layer of a structure according to arrangements described. In each layer, vias may have different diameters. The diameter of vias may be chosen based upon intended function and may, for example, spanning from 50 μm to 1.5 mm. For the case of smaller micro-vias below 0.1 mm diameter, it is necessary to consider how the via is to be plated, the plating type, plating thickness and core thickness. Thinner cores allow for laser drilling of smaller diameter vias (lower aspect ratio); further control regarding via diameter can be achieved as a result of the electroplating process adding material to the inner surface of a laser-drilled hole. Au coated vias (as well as the planar electrodes) can be additionally coated with other materials such as conducting polymers and carbon nanotubes to facilitate and improve signal to noise ratios.

By way of example, an implantation in which the biological sample comprises brain cells is described: based on brain cell dimensions, and interaction type under study, via diameter may necessarily vary. In particular, a minimum hole (via) diameter to allow nerves to grow from one layer to the other, can be determined by growing and differentiating iPS cells into dopaminergic neurons and identifying how much space such cells might need to connect between layers. Cell differentiation occurs in the FR4 layers. The interlayer nerves (in vias) can be imaged with upright and confocal laser scanning microscopy techniques and using scanning electron microscope (SEM). Such imaging can allow for identification of a nerve connection from one layer to the other from which a minimum via diameter for a particular cell type connection may be determined. Furthermore, the conductivity and connectivity of the vias is selected to allow for study of interlayer electrical communication between cells. Monitoring dopaminergic neurons firing from one layer to another by monitoring changes in electrical characteristics of a via can prove that monitoring interlayer electrical communication is possible in arrangements. Appropriate monitoring methods include: noise analysis, signal processing tools (to capture nerve signal propagation through both layers) and impedance spectroscopy to evaluate cell adhesion to the vias.

Various components and features may be added to arrangements. For example, layers may be enriched with cell growth factors to promote cellular growth from one layer to another. Arrangements may integrate biological sample culture components into the device. For example, a device may comprise one or more of: a source of cell culture media, a temperature sensor, heating stage and/or CO₂ valve to improve cell viability and self-sustenance. Such integration can allow for a cell culture environment, including cell culture media, temperature, CO₂ and/or fluidics to be accurately controlled via microcontrollers provided within the PCB module, or an external PCB arrangement.

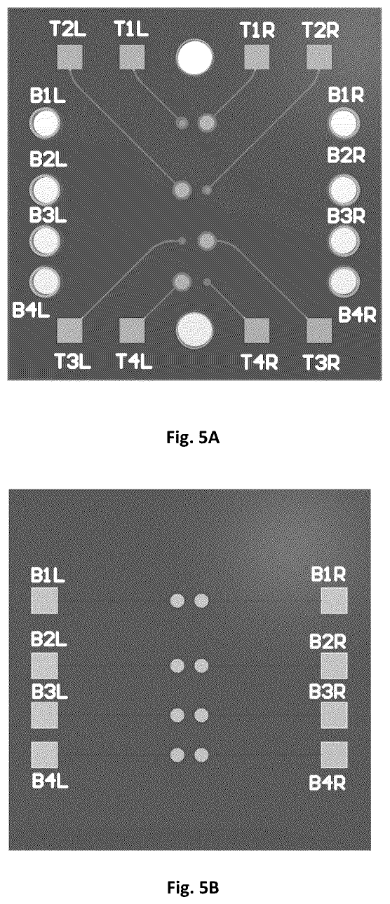

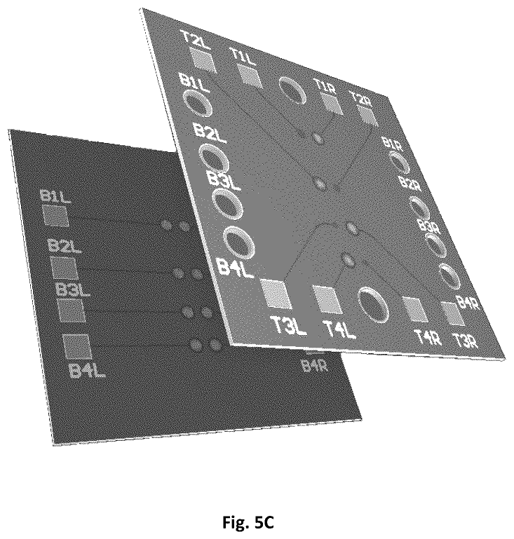

FIG. 4a is a representation of a possible 3 layer PCB stack which forms apparatus according to described arrangements. FIG. 4b is a representation of some components used to form a PCB stack such as that shown in FIG. 4a. As can be seen in FIG. 4a, an apparatus 10 can be formed from 3 layers of PCB 20a, 20b, 20c. The assembled apparatus is shown in FIG. 4a. Access vias 80, through which cells to be studied can be placed into appropriate chambers, can be seen on top layer 20c. Electrodes/connector points 100 connected via appropriate means 110 to electrodes 60 forming multi electrode arrays 60 in each cell chamber can be seen.

FIG. 4b shows the detail of each layer 20a, 20b, 20c. A via 70 connecting separate cell chambers can be seen. It will be understood that after assembly of a stack from layers 20a, 20b, 20c using, for example, adhesive components such as is shown 120, two chambers may be formed. One chamber will be formed between layers 20a and 20b. Another chamber is formed between layer 20b and 20c. Vias 80 in layers 20b and 20c align to form separate access from top layer 20c to each chamber. The chambers are physically linked by via 70. The inner surface of via 70 includes a conductive coating. In this example, gold plating.

### Applications

By way of example, apparatus may allow for the study of biological samples comprising brain cells. Apparatus such as those shown in FIG. 1 and FIG. 2 provides a mechanism to establishing in-vitro models of relevant brain microcircuits involving connected projection neurons and interneurons. The chamber and via arrangements allow for study of brain cell network activity with unprecedented technical options, for example, the chamber and via arrangement can allow for separation of interneurons from projection neurons. The chamber configuration of the apparatus can also permit establishment of brain circuit models with multiple inputs and relay neurons. In other words, the apparatus structure can allow construction of models of amygdala circuitry. Cellular sources for building neuronal networks in the chambers of the apparatus can be primary neurons as well as those differentiated from induced pluripotent stem cells.

Apparatus such as that shown schematically in FIG. 1 and FIG. 2 can facilitate efficient monitoring of strength and/or abundance of white matter connections correlates to quality of neuronal communication. Monitoring, decoding and intervening in brain communication, particularly how specific brain regions are wired together in circuits, can be achieved though appropriate use of apparatus according to arrangements. Arrangements which supply cell populations in chambers linked by vias can allow for readouts and electrical intervention in neuronal cells at the interception points of two or more well-defined cell populations.

Arrangements can use cells derived from living patients. The cells placed in the chambers of various described arrangements are such that they can be coupled in a bidirectional 3D structure, just as they might be in a human brain. As a result, arrangements can provide accurate modelling of the human brain in-vitro with in-vivo accuracy. Arrangements therefore promotes a significant reduction of animal usage in study of brain activity, together with a reduction of animal usage and patient side effects in the study of brain treatments.

Apparatus such as that shown in FIGS. 1 and 2 can support provision of experimental drug delivery and testing systems. Patient derived induced pluripotent stem cells (iPS cells) are differentiated into different cells and placed into multiple layers of a 3D sensor apparatus such as that shown in FIGS. 1 and 2. Using different pharmacological compounds, synaptic plasticity can be monitored both locally in a single layer of apparatus, where a unique and well defined cell population resides, or for example, throughout a set of interconnected PCB modules where multiple modelled “brain regions” are located.

It will be appreciated that arrangements formed as a PCB multilayer module can be constructed, and appropriate cells selected to be housed within chambers, to act as a module which mimics a selected portion of the brain, for example, the hippocampus. Modules mimicking various other brain regions, for example, frontal lobe, parietal lobe, temporal lobe, occipital lobe, cerebellum, brain stem, hypothalamus, pituitary gland and amygdala, may also be constructed. Integration of various PCB modules may allow for in-vitro modelling of an entire human brain.

Whilst described above in relation to modelling interactions between brain cells, it will be appreciated that the arrangements described also provide a device which supports various mechanisms to allow for study of biological samples. In a simple alternative to the brain cell application described above, the device may facilitate interaction between brain cells and other cells or for be used for the study of any excitable cells. By way of example, arrangements can be employed in cancer research to study tumour interaction with nearby neurons, by placing appropriate cells within chambers. It will be understood that such an arrangement provides the possibility to selectively record interactions between healthy and diseased tissue and concurrently monitor the electrical signalling in such scenarios.

Furthermore, the nature of the device, in which it is possible control and analyse interaction between one or more biological samples as they extend through the conductive channel, opens up various biological sample test modalities.

In addition, it will be appreciated that forming a biological sample housing from PCB can facilitate direct monitoring of the sample by integrating one or more electronic components into the PCB which forms at least part of the sample housing/chamber.

Although illustrative embodiments of the invention have been disclosed in detail herein, with reference to the accompanying drawings, it is understood that the invention is not limited to the precise embodiment and that various changes and modifications can be effected therein by one skilled in the art without departing from the scope of the invention as defined by the appended claims and their equivalents.

The term “comprising” whenever used in this document is intended to indicate the presence of stated features, integers, steps, components, but not to preclude the presence or addition of one or more other features, integers, steps, components or groups thereof.

The disclosure should not be seen in any way restricted to the embodiments described and a person with ordinary skill in the art will foresee many possibilities to modifications thereof.

The above described embodiments are combinable.

The following claims further set out particular embodiments of the disclosure.

1. A sensing apparatus comprising:
a chamber configured to receive a biological sample;
at least part of a wall of the chamber comprising a printed circuit board substrate;
the part of the wall comprising a printed circuit board substrate comprising an electrode configured to transmit or receive a signal to the biological sample, the electrode being coupleable to a sensing control unit located outside the chamber; and
a channel in communication with the chamber, the channel being dimensioned to allow the biological sample to extend through the channel, the channel comprising a conductive surface configured to transmit or receive a signal to at least a portion of the biological sample extendable through the channel, the conductive channel surface being coupleable to the sensing control unit located outside the chamber.

2. (canceled)

3. The sensing apparatus according to claim 1, wherein at least one of the substrate, the chamber, the electrode, the channel, and the conductive surface comprise a biocompatible material.

4. The sensing apparatus according to claim 1, wherein at least one wall of the chamber comprises a biological adhesion layer.

5. (canceled)

6. The sensing apparatus according to claim 1, wherein the conductive channel surface comprises a biological adhesion layer.

7. (canceled)

8. The sensing apparatus according to claim 4, wherein the biological adhesion layer comprises a material selected from the group consisting of: poly(lysine) (PL), poly(ornithine) (PO), poly(arginine), poly(ethylenimine) (PEI), poly-L-lysine (PLL), poly-D-lysine (PDL), poly-L-ornithine (PLO), and an extracellular cell matrix or a laminin coating.

9. The sensing apparatus according to claim 1, wherein at least one wall of the chamber is enriched with a cell growth factor.

10. The sensing apparatus according to claim 1, wherein the apparatus comprises: a second chamber configured to receive a second biological sample; at least part of a wall of the second chamber comprising a printed circuit board substrate; and the part of the wall of the second chamber comprising a printed circuit board substrate comprising a second chamber electrode configured to transmit or receive a second chamber signal to the second biological sample the second chamber electrode being coupleable to a sensing control unit located outside the second chamber, and wherein the channel connects the chamber and the second chamber.

11. The sensing apparatus according to claim 10, wherein the printed circuit board substrate comprising the at least part of a wall of the chamber and the printed circuit board substrate comprising the at least part of a wall of the second chamber are the same printed circuit board substrate.

12. (canceled)

13. The sensing apparatus according to claim 1, wherein the apparatus comprises: at least one further printed circuit board substrate located above or below the printed circuit board substrate comprising the at least part of a wall of the chamber, and wherein the chamber is formed between the printed circuit board substrate and the further printed circuit board substrate.

14. (canceled)

15. (canceled)

16. The sensing apparatus according to claim 1, wherein the apparatus comprises an access port associated with the chamber, the access port being configured to allow insertion of the biological sample into the chamber.

17. The sensing apparatus according to claim 10, wherein the apparatus comprises: an access port associated with each chamber, each access port being configured to allow insertion of a biological sample into the respective chamber.

18. The sensing apparatus according to claim 17, wherein the access port is configured to allow insertion of a biological sample into a respective chamber from an outer surface of the apparatus.

19. The sensing apparatus according to claim 1, wherein the channel comprises a via formed on the printed circuit board substrate.

20. (canceled)

21. (canceled)

22. The sensing apparatus according to claim 1, wherein the chamber and channel are in communication with each other and coupleable to a culture media source.

23. The sensing apparatus according to claim 22, further comprising a cell culture media circulation device; configured to circulate culture media through the chamber.

24. The sensing apparatus according to claim 1, wherein the channel of the sensing apparatus is coupleable with another sensing apparatus.

25. A method of forming sensing apparatus, the method comprising:
providing a chamber in which at least part of a wall of the chamber comprises a printed circuit board substrate, wherein the part of the wall comprising the printed circuit board substrate comprises an electrode configured to transmit or receive a signal to a biological sample locatable within the chamber, the electrode being coupleable to a sensing control unit located outside the chamber; and
providing a channel in communication with the chamber, the channel being dimensioned to allow the biological sample to extend through the channel, the channel comprising a conductive surface configured to transmit or receive a signal to at least a portion of the biological sample extendable through the channel, the conductive channel surface being coupleable to the sensing control unit located outside the chamber.

26. A sensing method comprising:
inserting a biological sample into the chamber of sensing apparatus according to claim 1; and
monitoring properties of the biological sample in the chamber and the channel using the electrode, conductive surface of the channel and the sensing control unit.

27. The sensing method according to claim 26, comprising:
electrically stimulating the biological sample in the chamber using the electrode and an electrical source; and
monitoring a response of the biological sample using a further electrode and the sensing control unit.

28. The sensing method according to claim 26, comprising:
chemically stimulating the biological sample in the chamber and monitoring a response of the biological sample using the electrode and the sensing control unit.
